(19) United States
(12) Patent Application Publication
(10) Pub. No.: US 2025/0261963 A1
(43) Pub. Date: Aug. 21, 2025

(54) Catheters, Catheter Assemblies, & Methods

(71) Applicant: Bard Peripheral Vascular, Inc., Franklin Lakes, NJ (US)

(72) Inventors: Matthew R. Casiraro, Tempe, AZ (US); Andrzej J. Chanduszko, Chandler, AZ (US)

(73) Assignee: Bard Peripheral Vascular, Inc., Franklin Lakes, NJ (US)

(21) Appl. No.: 18/702,979

(22) PCT Filed: Dec. 29, 2021
(86) PCT No.: PCT/US2021/065501

§ 371 Date: Apr. 19, 2024

(51) Int. Cl. A61B 17/3207; A61B 17/00

(52) U.S. Cl. CPC A61B 17/320758; A61B 2017/00893; A61B 2017/320716; A61B 2217/005; A61B 2217/007

(57) ABSTRACT

The present disclosure relates to catheters for use in removing an occlusion in a vessel. The catheter includes a cannula with a sidewall that defines a cannula lumen, and a distal portion, disposed at a distal end of the cannula, with a body that defines a body lumen. The catheter also includes a helix member that is rotatably disposed, at least partially, in the cannula lumen and body lumen. A longitudinally-oriented blade is attached to the helix member and at least a portion of the blade is intermittently exposed at the lateral opening. In operation, catheters according to the present disclosure generate negative pressure, which aspirates the occlusive material from the vessel through the catheter. In embodiments, while the occlusive material is being aspirated, the catheter causes the occlusive material to contact the clot fragmentation components, causing the occlusive material to break into smaller fragments.

### TECHNICAL FIELD

The present specification generally relates to catheters, catheter assemblies, and methods for aspirating, fragmenting and removing blood clots, fatty deposits, or other extractable material in medical procedures such as atherectomy and thrombectomy.

### BACKGROUND

Cardiovascular diseases are a leading cause of death worldwide. Vascular occlusions refer to blockages of blood vessels. Thrombosis is a specific type of vascular occlusion caused by a thrombus (i.e. a blood clot). Thrombi are formed from aggregated platelets, red blood cells, and fibrin proteins, but also may also include plaque and other blood-borne substances such as fat, cholesterol, and calcium.

Thrombi can form in both arteries and veins. Venous thrombosis leads to congestion of the affected part of the body, while arterial thrombosis affects the blood supply to tissue and can lead to ischemia or necrosis. Additional complications can arise when a piece of either an arterial or a venous thrombus breaks off. This piece of thrombus, called an embolus, can travel through the circulation and lodge somewhere else as an embolism.

Medical procedures such as thrombectomy can be used to remove thrombi from a blood vessel. One method of thrombectomy is mechanical aspiration. As a thrombus ages, it may become more fibrous and therefore more resistant to aspiration and mechanical fragmentation. This may result in blockages of existing aspiration devices due to a large clot burden. If the thrombectomy device is blocked, the procedure may be extended or an additional device may be required. When a thrombus is fragmented into smaller pieces, it is easier to transport the particles through the catheter and out of the patient without blockage of the device.

Accordingly, a need exists for devices to improve performance of occlusion removal by enhancing fragmentation and aspiration.

### BRIEF SUMMARY

Embodiments of the present disclosure are directed to catheter assemblies with enhanced aspiration and clot fragmentation components that assist in reducing the size of material removed from hollow anatomical structures, such as a vessel or the like. In operation, catheter assemblies according to the present disclosure generate negative pressure, which aspirates the occlusive material from the vessel through the catheter. In embodiments, catheter assemblies according to the present disclosure also operate to increase fragmentation of the occlusive material. While the occlusive material is being aspirated, the catheter assembly causes the occlusive material to contact the clot fragmentation components, causing the occlusive material to break into smaller fragments. The smaller fragments of the occlusive material are able to be aspirated through the cannula lumen of the catheter more effectively, thereby preventing blockage or clogging of the catheter according to the present disclosure.

According to one embodiment of the present disclosure, a catheter for use in removing an occlusion in a body lumen includes a cannula having a sidewall that defines a cannula lumen, and a distal portion, disposed at a distal end of the cannula, the distal portion comprising a body, said body defining a body lumen and said body further defining a lateral opening extending through the body and into the body lumen. The catheter also includes a helix member that is rotatably disposed, at least partially, in the cannula lumen and body lumen. A longitudinally-oriented blade is attached to the helix member and at least a portion of the blade is intermittently exposed at the lateral opening.

According to another embodiment of the present disclosure, a catheter assembly for use in removing an occlusion in a body lumen includes a cannula having a sidewall that defines a cannula lumen, and a distal portion, disposed at a distal end of the cannula. The distal portion has a body defining a body lumen, and a cylinder comprising a cylinder wall configured to define a cylinder opening. The catheter assembly includes a helix member rotatably disposed in the cannula lumen and the body lumen. The helix member has a distal terminal end and a rotational axis. The cylinder is coupled to the distal terminal end of the helix member. The cylinder opening longitudinally extends along the rotational axis. The cylinder also has a plurality of cantilevered blade members that extend inwardly from the cylinder wall toward the rotational axis.

According to another embodiment of the present disclosure, a method for removing occlusive material from a vessel, includes advancing a catheter to the occlusive material within the vessel; receiving the occlusive material within a lateral opening of a distal portion of the catheter; rotating a helix member positioned at least partially within the cannula lumen and the body lumen and rotatable with respect to a sidewall of the catheter, wherein a longitudinally-oriented blade is coupled to the helix and offset from a longitudinal axis of the catheter; fragmenting the occlusive material with the longitudinally-oriented blade as the occlusive material enters the lateral opening; and transporting the occlusive material from the lateral opening of the distal portion to a proximal portion of the catheter via the helix member.

According to another embodiment of the present disclosure, a method for removing occlusive material from a vessel, includes advancing a catheter assembly to the occlusive material within the vessel; rotating a helix member positioned at least partially within and rotatable with respect to a sidewall of the catheter assembly; receiving the occlusive material within a distal end opening of a distal portion of the catheter, wherein the distal end opening is coaxial with a cylinder opening defined by a cylinder wall of a cylinder, wherein the cylinder is connected to a distal terminal end of the helix member; fragmenting occlusive material with a plurality of cantilevered blade members that extend inwardly from the cylinder wall as the occlusive material enters the cylinder opening; and transporting the occlusive material from the cylinder opening to a proximal portion of the catheter assembly via the helix member.

Additional features and advantages of the technology disclosed in this disclosure will be set forth in the detailed description which follows, and in part will be readily apparent to those skilled in the art from the description or recognized by practicing the technology as described in this disclosure, including the detailed description which follows, the claims, as well as the appended drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a catheter including a helical member, according to one or more embodiments shown and described herein;

FIG. 2A schematically depicts the catheter of FIG. 1 with a lateral opening including one or more teeth, according to one or more embodiments shown and described herein;

FIG. 2B schematically depicts the catheter of FIG. 1 with a lateral opening including one or more teeth, according to one or more embodiments shown and described herein;

FIG. 2C schematically depicts the catheter of FIG. 1 with an alternative lateral opening and a distal end opening, according to one or more embodiments shown and described herein;

FIG. 2D schematically depicts the catheter of FIG. 1 with a maceration bar extending across the lateral opening, according to one or more embodiments shown and described herein;

FIG. 3A schematically depicts the helical member of FIG. 1 in isolation, according to one or more embodiments shown and described herein;

FIG. 3B depicts another embodiment of a helical member, according to one or more embodiments shown and described herein;

FIG. 4 schematically depicts use of a catheter to remove occlusive material from a body lumen, according to one or more embodiments described herein;

FIG. 5A schematically depicts the internal components of a catheter including a cylinder, according to one or more embodiments shown and described herein;

FIG. 5B schematically depicts the internal components of the catheter of FIG. 5A, having a lateral opening distal to the cylinder, according to one or more embodiments shown and described herein;

FIG. 5C schematically depicts an axial view of the cylinder positioned within the catheter of FIG. 5A, according to one or more embodiments shown and described herein;

FIG. 5D schematically depicts a perspective view of the cylinder of FIG. 5A in isolation, according to one or more embodiments shown and described herein;

FIG. 6 schematically depicts use of the catheter of FIG. 5A to remove occlusive material from a body lumen, according to one or more embodiments shown and described herein;

FIG. 7A schematically depicts the internal components of a catheter according to one or more embodiments shown and described herein;

FIG. 7B schematically depicts the internal components of a catheter according to one or more embodiments shown and described herein.

### DETAILED DESCRIPTION

Embodiments of the present disclosure are generally directed to catheter assemblies with enhanced aspiration and clot fragmentation components that assist in reducing the size of occlusions during removal from a body lumen. In operation, catheter assemblies according to the present disclosure generate negative pressure, which aspirates the occlusion from the body lumen through the catheter. In embodiments, catheter assemblies according to the present disclosure also operate to increase fragmentation of the occlusion. While the occlusive material is being aspirated, the catheter assembly causes the occlusive material to contact the clot fragmentation components, causing the occlusion to break into smaller fragments. The smaller fragments of the occlusive material are able to be aspirated through the cannula lumen of the catheter more effectively, thereby preventing blockage or clogging of the catheter according to the present disclosure.

The enhanced aspiration and clot fragmentation components of the present disclosure are structured to facilitate smaller pieces of occlusive material moving through the catheter, thereby providing predictable and continuous performance. Moreover, reducing the size of fragments of extractable material reduces stress on the helix member or other components of the catheter that may be caused by trying to aspirate large particles and enables better transport of the occlusion particles through the catheter. These and other embodiments of catheters are disclosed in greater detail herein with reference to the appended figures.

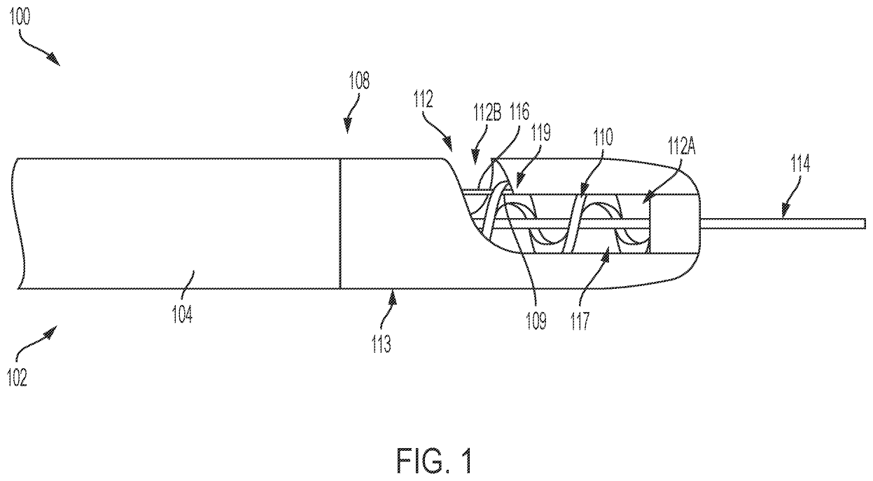

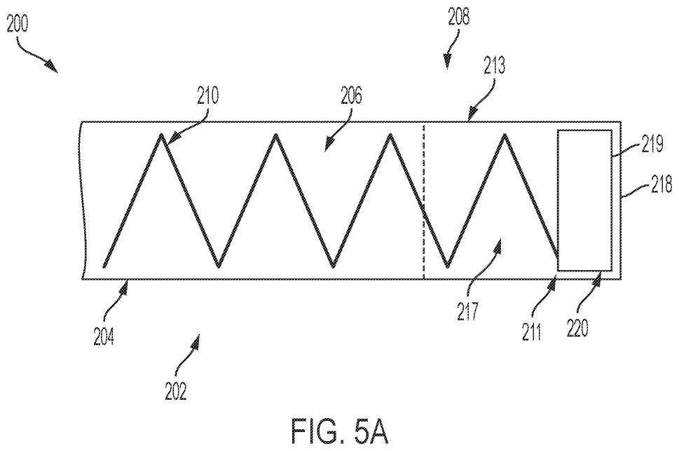

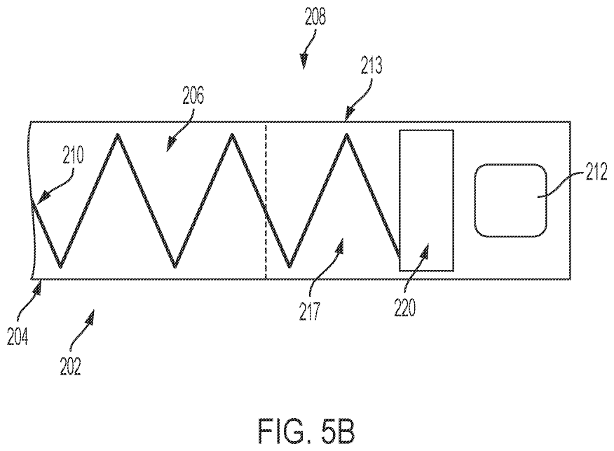

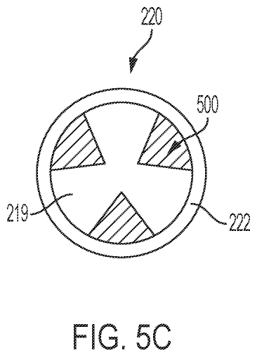

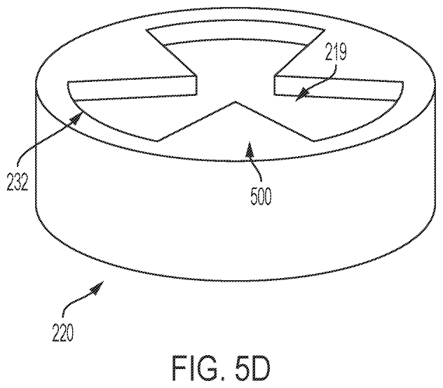

Now referring to FIG. 1, a catheter 100 is schematically depicted. In embodiments, the catheter 100 includes a cannula 102 having a sidewall 104. The sidewall 104 may define a cannula lumen 106 extending through the cannula 102, such as depicted in FIG. 5. The cannula 102 may be any material suitable for advancing through a vessel, such as, but not limited to, polyurethanes, polyamides, fluoropolymers, polyolefins, PVC, polyimides, polyetheretherketone, or the like. In embodiments, the sidewall 104 of the cannula 102 may be reinforced by any means suitable to preserve the flexibility of the cannula 102 while increasing stability, such as but not limited to, multilayered plastics, braided wire mesh, or the like.

The catheter 100 extends axially between a distal end and a proximal end. The catheter 100 may be sized to be advanced axially through a body lumen, such as a blood vessel, or the like. In some embodiments, a guidewire 114 may be inserted into a blood vessel, and the catheter 100 may be inserted into the blood vessel over the guidewire 114. For example, the guidewire 114 may extend axially through the cannula lumen 106. In some embodiments, there may not be a guidewire 114.

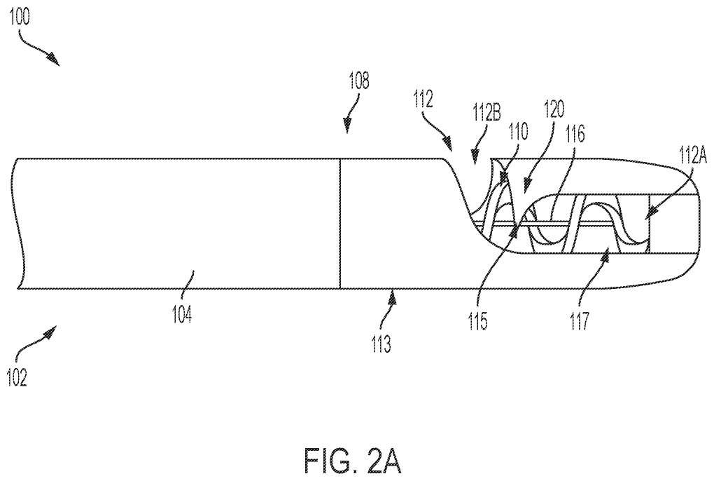

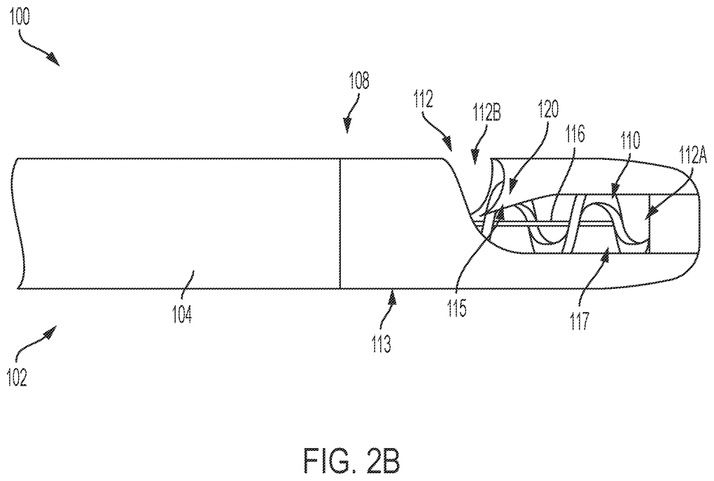

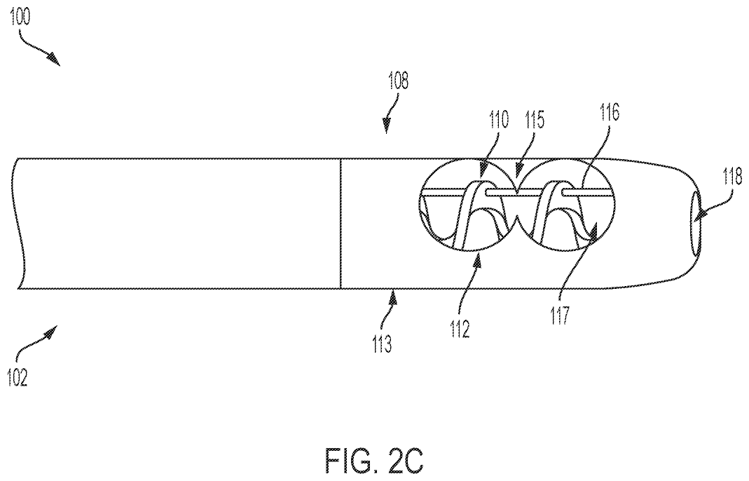

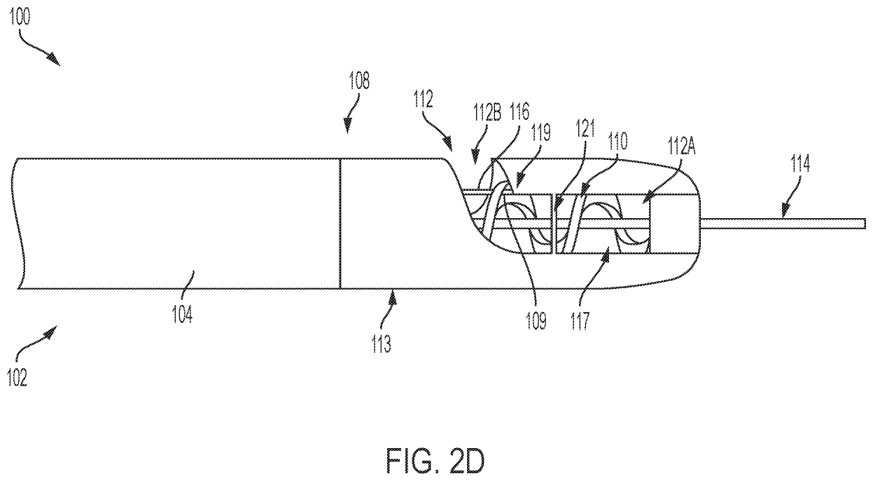

In embodiments, the catheter 100 includes distal portion 108 disposed at the distal end of cannula 102. In embodiments, the distal portion 108 may generally include a body 113. The body 113 may define a body lumen 117, which may be coaxial with the cannula lumen 106. The body 113, in some embodiments, may define a lateral opening 112 into the body lumen 117. In some embodiments, the lateral opening 112 may have a first section 112A that extends generally axially and a second section 112B that extends circumferentially, meeting at transition point 119 (e.g., to provide a generally L-shaped or J-shaped opening). In other embodiments, the lateral opening 112 can be generally triangular, with the width tapering towards the proximal end of the cannula 102. In some embodiments, the lateral opening 112 may be generally rectangular. Other shapes are contemplated and possible (e.g., circular, oval, or any polygonal or non-polygonal, regular, or irregular shape). For example, in embodiments, there may be more than one lateral opening. For example, multiple lateral openings may be disposed approximately diametrically from one another on the body 113. In embodiments, one or more portions of the lateral opening 112 extends along the body in the axial direction. In embodiments, one or more portions of the lateral opening 112 extend circumferentially about a longitudinal axis of the catheter 100. In some embodiments, the lateral opening 112 may be one or more circular holes. In embodiments, the circular holes can be arranged in axial succession. In some embodiments, the circular holes can be placed such that the circumferences of the holes overlap (such as depicted in FIG. 2C). The lateral opening 112 may be formed using conventional tools or techniques, such as drilling, milling, punching, erosion, water jet cutting, or the like. In some embodiments, the lateral opening 112 may be molded into the body during formation.

The distal portion 108 may be generally cylindrical and may taper toward the distal end. Tapering may improve advancement of the catheter 100 through a lumen. The distal portion 108 may also be made from different materials than cannula 102. Distal portion 108 may be any material suitable for advancing through a vessel and supporting clot fragmentation components, such as, but not limited to, metals (stainless steel, titanium, etc.) plastics, or composite materials.

Still referring to FIG. 1, the catheter 100 may also include a helix member 110 that is disposed in the cannula lumen 106 of catheter 100 and may extend into the body lumen 117 of the distal portion 108. For example, the helix member 110 may extend past the lateral opening 112 such that occlusive material entering through the lateral opening is met and directed proximally through the catheter 100 via the helix member 110. The helix member 110 may extend axially through catheter 100 so as to be substantially coaxial with the cannula lumen 106 and the body lumen 117. The helix member 110 may be coupled at a proximal end to a rotational actuator (e.g., a motor) which causes the helix member 110 to rotate about the longitudinal axis of the catheter 100 relative to the cannula 102. In some embodiments, the helix member 110 may be coupled to distal portion 108, such that rotation of the helix member 110 rotates the distal portion 108. For example, the helix member 110 may be welded, brazed, adhered, fastened, or otherwise fixedly attached to the distal portion. In some embodiments, the helix member 110 is not coupled to the distal portion 108, and the distal portion is not rotatable relative to the cannula 102. The helix member may generally be a wire or sheet wound into a helical shape (e.g., a helical spring shape). Accordingly, the helix member 110 may define a central lumen extending therethrough along the longitudinal axis of the catheter. Helix member 110 may be made from any suitable material, such as, but not limited to, stainless steel, titanium, or the like. In embodiments, the helix member 110 can be coated with any suitable biocompatible coatings, thereby reducing adhesion of blood to the helix member. Examples of suitable coatings may include, but are not limited to, hydrophilic coatings or pharmacologic coatings such as heparin, and the like.

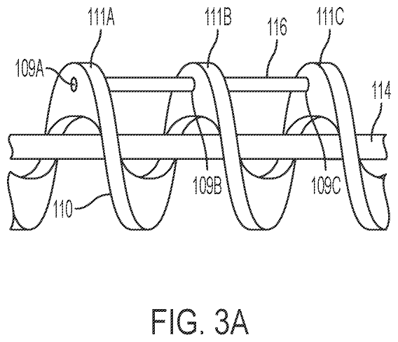

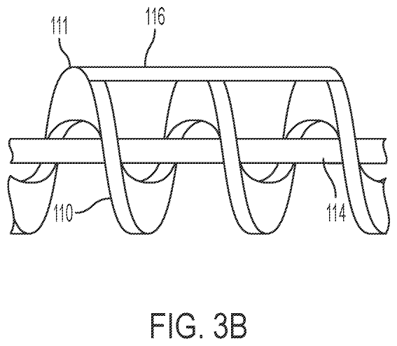

Still referring to FIG. 1, in embodiments, a longitudinally-oriented blade 116 may be attached to rotational helix member 110. Referring to FIG. 3A and 3B, in embodiments, longitudinally-oriented blade 116 may be welded, brazed, adhered, fastened, or otherwise fixedly attached to the rotational helix member 110. For example, in embodiments, longitudinally-oriented blade 116 may be attached to the rotational helix member 110 through one or more perforations 109 in rotational helix member 110. The longitudinally-oriented blade 116 may be oriented substantially parallel to a longitudinal axis of the catheter 100. A portion of the longitudinally-oriented blade 116 may be intermittently exposed at lateral opening 112, depicted in FIG. 1, during rotation of the rotational helix member 110. Intermittent exposure may refer to exposure of at least a portion of the longitudinally-oriented blade 116 at the lateral opening 112 depending on the rotational state of the rotational helix member 110. The longitudinally-oriented blade 116 may rotate with the helix member 110, helping to increase fragmentation and maceration of the occlusion as the occlusion particles enter through the lateral opening 112. The longitudinally-oriented blade 116 may be configured to cut tissue and/or occlusion particles. That is, in embodiments, the longitudinally-oriented blade 116 provides an additional cutting mechanism in addition to the rotational helix member 110 itself. In some embodiments, the longitudinally-oriented blade 116 may define a sharp cutting edge. In other embodiments, the longitudinally-oriented blade 116 may be substantially blunt-edged. In embodiments, the longitudinally-oriented blade 116 is formed of a metallic wire or ribbon. For example, longitudinally-oriented blade 116 may be any material suitable for attachment to helix member 110 and fragmentation of the occlusion, such as, but not limited to, stainless steel, titanium, and the like. In embodiments, a plurality of blades may attached to the helix and spaced approximately equidistant from each other.

Referring now to FIGS. 2A-2C, the catheter 100 is depicted without the guidewire 114 and with the lateral opening 112 including one or more additional features or elements (e.g., corners, grooves, sharpened edges, bumps, protrusions, cavities, and the like) to further facilitate fragmentation of the occlusive material as it is drawn into the body lumen 117 through the lateral opening 112. For example, an edge of the lateral opening 112 may be formed as a cutting edge 115, thus ensuring improved fragmentation of the material aspirated by the catheter 100. In embodiments, the cutting edge 115 may be aligned with the body 113 of the distal portion 108. In embodiments, the cutting edges 115 may be disposed inwards or recessed relative to the body 113. In embodiments, the cutting edges 115 may have a structured shape, e.g. zigzagged. In embodiments, the cutting edge 115 may protrude into lateral opening 112. For example, extending from an edge of the lateral opening may be one or more teeth 120 defining at least a portion of the cutting edge 115. As depicted in FIG. 2A, the one or more teeth 120 may extend in a circumferential direction across the lateral opening 112. In other embodiments, such as depicted in FIG. 2B, the one or more teeth 120 may extend along an axial direction across the lateral opening 112. In other embodiments, there may be a combination of the laterally and circumferentially extending teeth. Although in each embodiment, there is only depicted a single tooth, there may be a plurality of teeth, e.g., two or more teeth, three or more teeth, etc. Additionally, wherein the lateral opening forms a “J” or “L” shape, the one or more teeth may be at a transition point 119, depicted in FIG. 1, between the first section 112A and the second section 112B. The cutting edge 115 of the one or more teeth 120 may be curved or straight.

As noted above, in some embodiments, the lateral opening 112 may include one or more circular holes. In embodiments, the circular holes can be arranged in axial succession. In some embodiments, as depicted in FIG. 2C, the circular holes can be placed such that the circumferences of the holes overlap. In embodiments, the overlap of the holes can be configured to form one or more cutting edges 115. In some embodiments, the distal portion 108 may further include a distal end opening 118. In embodiments, the distal end opening 118 may be sized and shaped to provide entry and/or egress into the body lumen 117 of the body 113. In embodiments, distal end opening 118 may be coaxial with body lumen 117 and cannula lumen 106. In embodiments, as occlusive material is drawn through lateral opening 112 and distal end opening 118 into the body lumen 117, the occlusive material is fragmented by longitudinally-oriented blade 116 and/or the helix member 110. In some embodiments, distal end opening 118 may allow for additional aspiration of occluding material.

In embodiments, such as depicted in FIG. 2D, the catheter 100 may include a maceration bar 121, extending across the lateral opening 112. In embodiments, the maceration bar 121 may be welded, brazed, adhered, fastened, or otherwise fixedly attached to the body 113. In embodiments, the maceration bar 121 may be molded into the body 113 during formation. In embodiments, the maceration bar 121 may extend circumferentially about a longitudinal axis of the catheter 100. The maceration bar 121 may be configured to cut tissue and/or occlusion particles. That is, in embodiments, the maceration bar 121 facilitates fragmentation of the occlusive material as it is drawn into the body lumen 117 through the lateral opening 112. Stated another way, as occlusive material is drawn through lateral opening 112, the occlusive material is fragmented by the maceration bar 121. The fragmented material may be then aspirated into the body lumen 117, where the longitudinally-oriented blade 116 and/or the helix member 110 may facilitate further fragment the occlusive material. In some embodiments, the maceration bar 121 may define a sharp edge to assisting in cutting and/or fragmentation. In other embodiments, the maceration bar 121 may be substantially blunt-edged. The maceration bar 121 may be included in any of the embodiments described herein.

FIG. 3A schematically depicts an embodiment of a distal portion of the rotational helix member 110 and the guidewire 114 in isolation from the cannula 102 and the distal portion 108. As depicted and noted above, the longitudinally-oriented blade 116 may be attached to a distal portion of the rotational helix member 110 that is positioned in the distal portion 108 of the catheter 100. In embodiments, the longitudinally-oriented blade 116 may be attached to one or more loops 111 of the rotational helix member (e.g., two or more loops, three or more loops etc.) such that the longitudinally oriented blade may extend across an entirety of the lateral opening in the axial direction. For example, in the depicted embodiment, the longitudinally-oriented blade 116 is coupled to three consecutive loops 111a, 111b, 111c. As noted above, in embodiments, the longitudinally-oriented blade 116 may be attached to the rotational helix member 110 through a perforation in rotational helix member 110. Accordingly, each of the consecutive loops 111a, 111b, 111c, may each include a perforation 109a, 109b, 109c, or opening sized to receive the longitudinally-oriented blade 116. The perforations may be positioned between an inner diameter and an outer diameter of the rotational helix member 110, such that the longitudinally-oriented blade 116 is completed surround by material of the rotational helix member 110 at each perforation through which it extends.

As depicted in FIG. 3B, in embodiments, longitudinally-oriented blade 116 may instead attach to a circumferential periphery of rotational helix member 110. That is, the longitudinally-oriented blade 116 may connect to the outer perimeter of the helix member 110. As in the embodiment above, the longitudinally-oriented blade 116 may be coupled to the helix member 110 at one or more locations. For example, the longitudinally-oriented blade 116 may be coupled to one or more loops 111 of the helix member 110 (e.g., two or more loops, three or more loops, etc.) such that the longitudinally-oriented blade may extend across an entirety of the lateral opening in the axial direction. In some embodiments, the longitudinally-oriented blade 116 may not be coupled to consecutive loops and may be coupled to non-consecutive loops, while simply extending past any intervening loops.

As noted above, the helix member 110 may be operatively coupled to a motor (not shown). Rotation of the helix may be controlled by user input. In embodiments, the user input may include a foot pedal, push-button, slider, toggle, switch, touch screen, or the like that can be actuated by a user. The rotation of the helix member 110 generates negative pressure in the catheter 100. In embodiments, the helix member rotates from about 10,000 to about 200,000 rpm, though other rotational speeds are contemplated and possible based on the particular application. The negative pressure produced by the rotation of the helix member 110 is strong enough to aspirate fragments of occlusive material into the body lumen 117 through the lateral openings 112. For example, the aspiration rate may be about 45 ml/min or more, about 75 ml/min or more, or about 130 ml/min or more, though other rates are contemplated and possible. The negative pressure generated by the rotation of rotational helix member 110 carries the fragmented material through the body lumen 117, through the cannula lumen 106 from the distal end of the catheter 100 to the proximal end and into a collection receptacle (not shown). In embodiments, an external vacuum (not shown) may be added to increase the negative pressure inside the catheter 100.

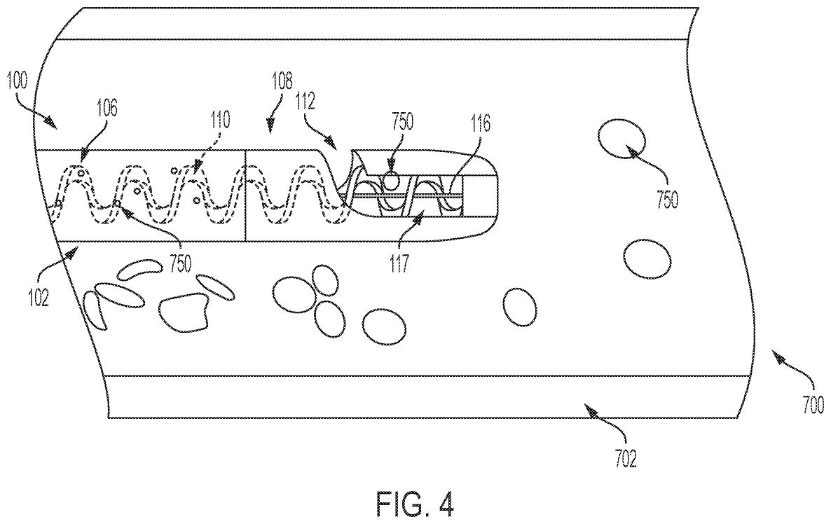

FIG. 4 schematically illustrates aspiration of occlusive material 750 by the catheter 100. It is noted that while the embodiment of FIG. 1 is illustrated, any of the embodiments may be used. As shown in FIG. 4, when the distal portion 108 encounters occlusive material 750 within a vessel 700 (e.g. a vein, an artery, etc.), the occlusive material 750 can be aspirated through the catheter 100 via negative pressure generated by rotation of the rotational helix member 110. In embodiments, the catheter 100 can be positioned in a vessel 700 and advanced in an axial direction, substantially parallel to vessel wall 702. As noted above, in embodiments, when the distal portion 108 encounters the occlusive material 750, the rotation of the rotational helix member 110 generates negative pressure within the body lumen 117 and cannula lumen 106. In some embodiments, as depicted, the occlusive material 750 may be aspirated into the body lumen 117 through the lateral opening 112 (or an end opening as described above). As occlusive material 750 is drawn into the body lumen 117 through the lateral opening 112, the longitudinally-oriented blade 116 assists in fragmenting the occlusive material 750 into smaller particle sizes. The fragmented material is then transported away from lateral opening 112 in a proximal direction through catheter 100 via helix member 110.

Referring now to FIGS. 5A-D, another embodiment of a catheter 200 is schematically depicted, showing internal components. The catheter 200 is similar to catheter 100 as described above. In particular, the catheter 200 includes a cannula 202 having a sidewall 204. The sidewall 104 may define a cannula lumen 206 extending through the cannula 202. The cannula 202 is substantially the same as the cannula 102, as described above.

In embodiments, the catheter 200 includes a distal portion 208 disposed at the distal end of the cannula 202. In embodiments, the distal portion 208 may generally include a body 213. The body 213 may define a body lumen 217, which may be coaxial with the cannula lumen 206. The distal portion 208 is substantially similar to the distal portion 108, described above. For example, the distal portion 208 may be generally cylindrical. Additionally, though not depicted, the distal portion 208 may taper toward the distal end. Tapering may improve advancement of the catheter 200 through a vessel. The distal portion 208 may also be made from different materials than the cannula 202. In some embodiments, the distal portion 208 may further include a distal end opening 218. In embodiments, the distal end opening 218 may be sized and shaped to provide entry and/or egress into the body lumen 217 of the body 213. In embodiments, the distal end opening 218 may be coaxial with the body lumen 217 and the cannula lumen 206. In some embodiments, the distal end opening 218 may allow for aspiration of occluding material. It is noted that while the present embodiment, only depicts a distal end opening 218, a lateral opening, such as in the embodiments described above, may be included without departing from the scope of the present disclosure.

In the present embodiment, the distal portion 208 may include a cylinder 220, as further illustrated in FIGS. 5C and 5D, positioned within the body 213. In embodiments, the cylinder 220 may be a hollow cylinder, with a cylinder wall 222 defining a cylinder opening 219. In embodiments, the cylinder opening 219 may be coaxial with the body lumen 217 and the cannula lumen 206. In embodiments, cylinder opening 219 may be coaxial with distal end opening 218. In some embodiments, such as depicted in FIG. 5B, the body 213 of the distal portion 208 may in place of or in addition to the distal end opening 218 may define a lateral opening 212 into the body lumen 217. As noted above, the lateral opening 212 is substantially similar to the lateral opening 112, described in detail above. In some embodiments, the lateral opening 212 may allow for additional aspiration of occluding material. In such embodiments, the cylinder 220 may be positioned proximally to the lateral opening 212.

The cylinder 220 may include one or more features to aid in macerating occlusive material as it enters the catheter 200. For example, and with reference to FIG. 5C and 5D, the cylinder 220 may include a plurality of cantilevered blade members 500 that extend inwardly from the cylinder wall 222 toward a longitudinal axis (or the rotational axis) of the catheter 200. In embodiments, the cylinder 220 may include a single cantilevered blade member 500. In embodiments, the blade members 500 may be triangularly shaped, though other shapes are contemplated and possible (e.g., rectangular, square, leaf, etc.). In some embodiments, the blade members 500 may be staggered longitudinally along the cylinder wall 122 so that at least one blade member is positioned proximally to another blade member. In embodiments, the blade members 500 may be randomly staggered along cylinder wall 222 or the blade members 500 may be staggered at regular intervals along the cylinder wall 222. In embodiments, the blade members 500 may be angled in the proximal or distal directions. Cantilevered blade members 500 may fragment the occlusive material as it is aspirated into the body lumen 217. It is noted that the cylinder 220 and cantilevered blade members 500 may be included in any of the embodiments described herein. It is further contemplated that though the plurality of cantilevered blade members 500 are illustrated as extending inwardly from the cylinder wall 222, in some embodiments, the plurality of cantilevered blade members 500 may extend outwardly from the cylinder wall 222.

Referring again to FIG. 5A, in embodiments, the catheter 200 may also include a helix member 210 that is disposed in the cannula lumen 206 and may extend into the body lumen 217 of the distal portion 208. The helix member 210 is substantially the same as the helix member 110 described in detail above. The helix member 210 may extend axially through catheter 200 so as to be substantially coaxial with the cannula lumen 206 and the body lumen 217. The helix member 210 may be coupled at a proximal end to a rotational actuator (e.g., a motor) which causes the helix member 210 to rotate about the longitudinal axis of the catheter 200 relative to the cannula 202.

In embodiments, cylinder 220 may be coupled to a distal terminal end 211 of rotational helix member 210 via any conventional joining technique (e.g., loop connectors, brazing, welding, adhesives, or the like). Accordingly, as the rotational helix member 210 rotates, the cylinder 220 would also rotate, enhancing fragmentation of the occlusive material as it enters the body lumen 217 through distal end opening 218. In some embodiments, it is contemplated that the cylinder 220 may be coupled to the body 213 (e.g., via, brazing, welding, or the like), such that the cylinder 220 rotates with the body 220. In either case, as the cylinder 220 rotates, particles going through the cylinder may be macerated and particularized via the plurality of cantilevered blade members 500.

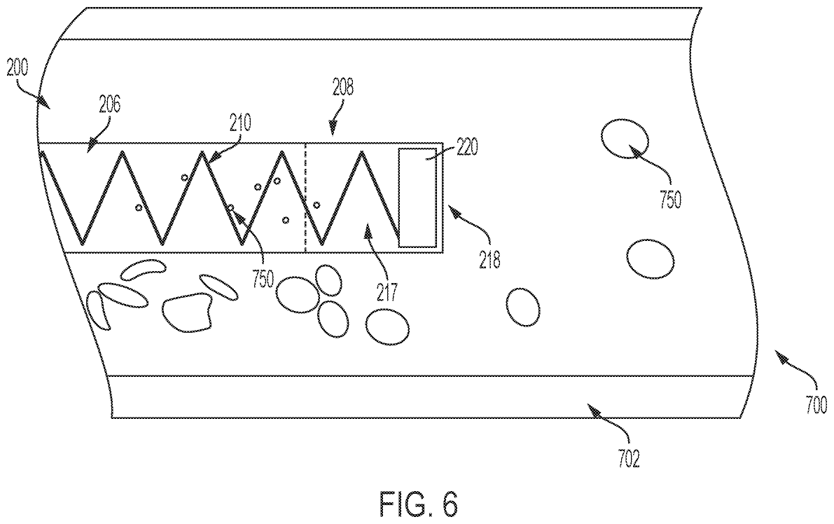

FIG. 6 schematically illustrates aspiration of occlusive material 750 by the catheter 200. It is noted that while the embodiment of FIG. 5A is illustrated; any of the embodiments may be used. As shown in FIG. 6, the catheter 200 can be positioned in a vessel (e.g. a vein, an artery, etc.) 700 and advanced in an axial direction, substantially parallel to a vessel wall 702 until the distal portion 208 encounters occlusive material 750. As noted above, in embodiments, when the distal portion 208 encounters occlusive material 750, the rotation of the rotational helix member 210 generates negative pressure within the body lumen 217 of the distal portion 208. In embodiments, the occlusive material 750 may be aspirated into the body lumen 217 of the distal portion 208 through distal end opening 218 (or the lateral side opening 212, where include). As occlusive material 750 is drawn into the body lumen 217, the occlusive material 750 moves proximally toward the cylinder 220 positioned in the body 213 of the distal portion 208. The plurality of cantilevered blade members 500 extending inwardly from cylinder wall 222 of cylinder 220 assists in fragmenting the occlusive material 750 into smaller particle sizes. The fragmented material is then transported away from the cylinder 220 in a proximal direction through the catheter 200 via helix member 210.

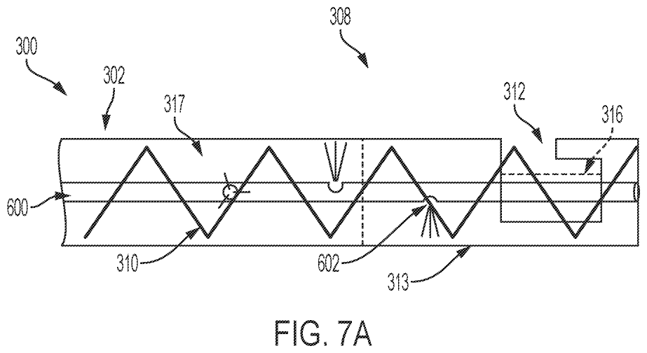

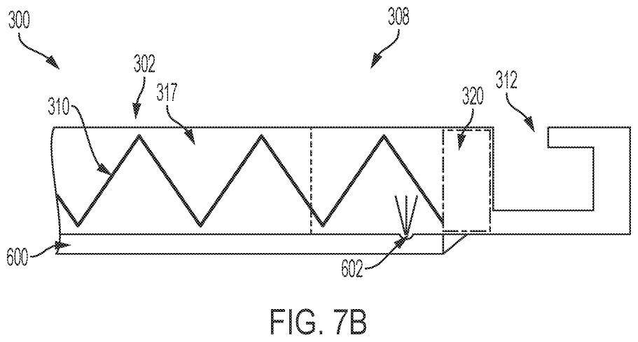

Referring now to FIGS. 7A and 7B, another embodiment of a catheter 300 is schematically depicted showing internal components. The catheter 300 is similar to catheters 100 and 200 as described above. In particular, the catheter 300 includes a cannula 302 that is substantially the same as the cannula 102 and the cannula 202, as described in detail above.

In embodiments, the catheter 300 includes a distal portion 308 disposed at the distal end of a cannula. In embodiments, the distal portion 308 may generally include a body 313. The body 313 may define a body lumen 317. The distal portion 308 is substantially similar to the distal portion 108 and the distal portion 208 as described in detail above. The distal portion 308 may be generally cylindrical. As in embodiments above, though not shown, the distal portion 308 may taper toward the distal end. In embodiments, the catheter 300 may also include a helix member 310 that is disposed in the catheter 300 and may extend into the body lumen 317 of the distal portion 308. The helix member 310 is substantially the same as the helix member 110 and the helix member 210 described in detail above. The helix member 310 may extend axially through the catheter 300 so as to be substantially coaxial with the body lumen 317. The helix member 310 may be coupled at a proximal end to a rotational actuator (e.g., a motor) which causes the helix member 310 to rotate about the longitudinal axis of the catheter 300. In some embodiments, the body 313 of the distal portion 308 may define a lateral opening 312 into the body lumen 317. The lateral opening 312 is substantially similar to the lateral opening 112, described in detail above. In some embodiments, the lateral opening 312 may allow for aspiration of the occlusive material as it is drawn into the body lumen 317 through the lateral opening 312.

In embodiments, the catheter 300 may further include an irrigation assembly 600 configured to dispense or pump fluid through a plurality of fluid delivery ports 602 into the body lumen 317. In embodiments, the fluid is pumped at a rate of about 60 cc/min, though other rates are contemplated and possible. Fluid delivery ports 602 are configured to deliver pressurized fluid into the body lumen 317. In embodiments, the fluid can be any suitable sterile liquid. Examples of suitable liquids may include, but are not limited to, saline, water, thrombolytic agents, and the like. In embodiments, the fluid is pressurized at about 10,000 psi, though other rates are contemplated and possible. Negative pressure generated from the rotation of the helix member 310 aspirates the fluid and fragmented occlusive material into a proximal portion of catheter 100 and to a collection receptacle (not shown).

In embodiments, as depicted in FIG. 7A the irrigation assembly 600 is disposed in the body lumen 317, so as to be positioned coaxially within the helix member 310. In embodiments, the irrigation assembly 600 may be coupled to the distal portion 308 (e.g., via, brazing, welding, or the like). The irrigation assembly 600 may extend axially through the catheter 300 along the longitudinal axis of the catheter 300. In embodiments, as an occlusion is aspirated through the lateral opening 312, the fluid delivery ports 602 deliver fluid into the body lumen 317, causing increased fragmentation of the occlusion. In embodiments, the fluid delivery ports 602 may be disposed to deliver fluid at a desired angle. For example, fluid delivery ports 602 may deliver fluid orthogonally or non-orthogonally to the irrigation assembly. In embodiments, the fluid may be delivered through the fluid delivery ports 602 continuously. In embodiments, the fluid may be delivered through the fluid delivery ports 602 in pulses. In embodiments, the catheter 300 may include a longitudinally-oriented blade 316. The longitudinally oriented-blade 316 is substantially similar to the longitudinally-oriented blade 116 described in detail above. The longitudinally-oriented blade is coupled to rotational helix member 310 as described above. As an occlusion is aspirated through the lateral opening 312, the occlusive material encounters longitudinally-oriented blade 316, causing increased fragmentation of the clot. Then, the fluid delivery ports 602 may deliver fluid into the body lumen 317, causing further increased fragmentation of the occlusion. Negative pressure generated from the rotation of the helix member 310 aspirates the fluid and fragmented occlusive material into a proximal portion of catheter 300.

In some embodiments, the irrigation assembly 600 may be coupled to an external circumferential periphery of distal portion 308 (e.g., via adhesives, welding, brazing, or the like), such as illustrated in FIG. 7B. In this embodiment, the fluid delivery ports may extend through the distal portion 308 to inject fluid toward the longitudinal axis of the catheter 300. In some embodiments, the longitudinally-oriented blade, such as depicted in FIG. 7A may be included. However, in some embodiments, distal portion 308 includes a cylinder 320. The cylinder 320 is substantially similar to the cylinder 220, described in detail above. The cylinder 320 may include one or more features, such as the plurality of blade members described above, to aid in macerating occlusive material as it enters the catheter 300. As an occlusion is aspirated into catheter 300, the occlusive material encounters cylinder 320, causing increased fragmentation of the clot. Then, the fluid delivery ports 602 may deliver fluid into the body lumen 317, causing further increased fragmentation of the occlusion. Negative pressure generated from the rotation of the helix member 310 aspirates the fluid and fragmented occlusive material into a proximal portion of catheter 100. The irrigation assembly 600 may be included in any of the embodiments described herein.

Embodiments can be further described with reference to the following numerical clauses:
1. A catheter comprising: a cannula comprising a sidewall defining a cannula lumen; a distal portion, disposed at a distal end of the cannula, the distal portion comprising a body, said body defining a body lumen and said body further defining a lateral opening extending through the body and into the body lumen; a helix member positioned at least partially within (the body and the cannula) and rotatable with respect to the sidewall; and a longitudinally-oriented blade, attached to said helix member, wherein at least a portion of the blade is intermittently exposed at the lateral opening. The longitudinally-oriented blade may be configured to cut tissue and/or occlusion particles.
2. The catheter of the preceding clause, wherein the longitudinally-oriented blade is coupled to three or more loops of the helix member.
3. The catheter of any preceding clause, wherein the body comprises one or more cutting edges disposed at the lateral opening.
4. The catheter of any preceding clause, wherein the cutting edge is configured to extend across a portion of the lateral opening.
5. The catheter of any preceding clause, wherein the body defines a distal end opening.
6. The catheter of any preceding clause, further comprising an irrigation assembly, the irrigation assembly comprising a plurality of fluid delivery ports configured to dispense fluid into the body lumen and/or the cannula lumen.
7. The catheter of the preceding clause, wherein the fluid comprises saline or thrombolytic agents
8. The catheter of any preceding clauses 6 or 7, wherein the irrigation assembly is coupled to a circumferential periphery of the distal portion.
9. The catheter of any of preceding clauses 6-8, wherein the irrigation assembly is disposed in the cannula lumen. The catheter assembly of clauses 1 to 9 may also have features of the catheter assembly of clauses 10 to 15.
10. A catheter assembly comprising, a cannula comprising a sidewall defining a cannula lumen; a distal portion, disposed at a distal end of the cannula, the distal portion comprising: a body, said body defining a body lumen, and a cylinder comprising a cylinder wall configured to define a cylinder opening; a helix member rotatably disposed in the cannula lumen and the body lumen, the helix member having a distal terminal end and a rotational axis; wherein said cylinder is coupled to the distal terminal end of the helix member and said cylinder opening extends longitudinally along the rotational axis; and a plurality of cantilevered blade members that extend inwardly from the cylinder wall toward the rotational axis.
11. The catheter assembly of the preceding clauses 10, wherein the body defines a lateral opening in the distal portion.
12. The catheter assembly of the preceding clause 11, wherein the body comprises one or more cutting edges disposed at the lateral opening.
13. The catheter assembly of any preceding clauses 10 to 12, further comprising an irrigation assembly, said irrigation assembly comprising a plurality of fluid delivery ports configured to dispense fluid into the body lumen.
14. The catheter assembly of the preceding clause 13, wherein the irrigation assembly is coupled to a circumferential periphery of the distal portion.
15. The catheter assembly of any preceding clauses 12-14, wherein the fluid comprises saline and/or thrombolytic agents. The catheter assembly of clauses 10 to 15 may also have features of the catheter of clauses 1 to 10.
16. A method for removing occlusive material from a vessel, the method comprising: advancing a catheter to the occlusive material within the vessel; receiving the occlusive material within a lateral opening of a distal portion of the catheter; rotating a helix member positioned at least partially within the cannula lumen and the body lumen and rotatable with respect to a sidewall of the catheter, wherein a longitudinally-oriented blade is coupled to the helix and offset from a longitudinal axis of the catheter; fragmenting the occlusive material with the longitudinally-oriented blade as the occlusive material enters the lateral opening; and transporting the occlusive material from the lateral opening of the distal portion to a proximal portion of the catheter via the helix member.
17. The method of any preceding clause wherein the distal portion comprises one or more cutting edges disposed at the lateral opening.
18. The method of any preceding clause, wherein at least a portion of the longitudinally-oriented blade is intermittently exposed at the lateral opening.
19. The method of any preceding clause, further comprising dispensing fluid into a body lumen of the distal portion using an irrigation assembly, wherein said irrigation assembly comprises a plurality of fluid delivery ports.
20. A method for removing occlusive material from a vessel, the method comprising: advancing a catheter assembly to the occlusive material within the vessel; rotating a helix member positioned at least partially within and rotatable with respect to a sidewall of the catheter assembly; receiving the occlusive material within a distal end opening of a distal portion of the catheter, wherein the distal end opening is coaxial with a cylinder opening defined by a cylinder wall of a cylinder, wherein the cylinder is connected to a distal terminal end of the helix member; fragmenting occlusive material with a plurality of cantilevered blade members that extend inwardly from the cylinder wall as the occlusive material enters the cylinder opening; and transporting the occlusive material from the cylinder opening to a proximal portion of the catheter assembly via the helix member.
21. The method of any preceding clause, wherein the distal portion defines a lateral opening, positioned distal to the cylinder.

It should now be understood that embodiments of the present disclosure pertain to catheters for use in removing an occlusion in a body lumen, which include a cannula having a sidewall that defines a lumen, and a distal portion. The distal portion includes openings to allow for occlusion aspiration. The catheter also includes a helix member that is rotatably disposed in the cannula lumen. The distal portion openings may be lateral or distal. Lateral openings may extend through the sidewall to the cannula lumen. The distal end portion of the helix member may include a longitudinally orientated blade attached to a circumferential periphery of the helix member. The blade may be intermittently exposed at the lateral opening.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter comprising:
a cannula comprising a sidewall defining a cannula lumen;
a distal portion, disposed at a distal end of the cannula, the distal portion comprising a body, said body defining a body lumen and said body further defining a lateral opening extending through the body and into the body lumen;
a helix member positioned at least partially within the cannula lumen and the body lumen and rotatable with respect to the sidewall; and
a longitudinally-oriented blade, attached to said helix member, wherein at least a portion of the longitudinally-oriented blade is intermittently exposed at the lateral opening.

2. The catheter of claim 1, wherein the longitudinally-oriented blade is coupled to three or more loops of the helix member.

3. The catheter of claim 1, wherein the body comprises one or more cutting edges disposed at the lateral opening.

4. The catheter of claim 3, wherein the cutting edge is configured to extend across a portion of the lateral opening.

5. The catheter of claim 1, wherein the body defines a distal end opening.

6. The catheter of claim 1, further comprising an irrigation assembly, the irrigation assembly comprising a plurality of fluid delivery ports configured to dispense fluid into the body lumen.

7. The catheter of claim 6, wherein the fluid comprises saline or thrombolytic agents.

8. The catheter of claim 6, wherein the irrigation assembly is coupled to a circumferential periphery of the distal portion.

9. The catheter of claim 6, wherein the irrigation assembly is disposed in the cannula lumen.

10. A catheter assembly, comprising:
a cannula comprising a sidewall defining a cannula lumen;
a distal portion, disposed at a distal end of the cannula, the distal portion comprising:
a body, said body defining a body lumen; and
a cylinder comprising a cylinder wall configured to define a cylinder opening;
a helix member rotatably disposed in the cannula lumen and the body lumen, the helix member having a distal terminal end and a rotational axis;
wherein said cylinder is coupled to the distal terminal end of the helix member and said cylinder opening extends longitudinally along the rotational axis; and
a plurality of cantilevered blade members that extend inwardly from the cylinder wall toward the rotational axis.

11. The catheter assembly of claim 10, wherein the body defines a lateral opening in the distal portion.

12. The catheter assembly of claim 11, wherein the body comprises one or more cutting edges disposed at the lateral opening.

13. The catheter assembly of claim 10, further comprising an irrigation assembly, said irrigation assembly comprising a plurality of fluid delivery ports configured to dispense fluid into the body lumen.

14. The catheter assembly of claim 13, wherein the irrigation assembly wherein the irrigation assembly is coupled to a circumferential periphery of the distal portion.

15. The catheter assembly of claim 13, wherein the fluid comprises saline or thrombolytic agents.

16. A method for removing occlusive material from a vessel, the method comprising:
advancing a catheter to the occlusive material within the vessel;
receiving the occlusive material within a lateral opening of a distal portion of the catheter;
rotating a helix member positioned at least partially within the cannula lumen and the body lumen and rotatable with respect to a sidewall of the catheter, wherein a longitudinally-oriented blade is coupled to the helix member and offset from a longitudinal axis of the catheter;
fragmenting the occlusive material with the longitudinally-oriented blade as the occlusive material enters the lateral opening; and
transporting the occlusive material from the lateral opening of the distal portion to a proximal portion of the catheter via the helix member.

17. The method of claim 16, wherein the distal portion comprises one or more cutting edges disposed at the lateral opening.

18. The method of claim 16, wherein at least a portion of the longitudinally-oriented blade is intermittently exposed at the lateral opening.

19. The method of claim 16, further comprising dispensing fluid into a body lumen of the distal portion using an irrigation assembly, wherein said irrigation assembly comprises a plurality of fluid delivery ports.

20. A method for removing occlusive material from a vessel, the method comprising:
advancing a catheter assembly to the occlusive material within the vessel;
rotating a helix member positioned at least partially within and rotatable with respect to a sidewall of the catheter assembly;
receiving the occlusive material within a distal end opening of a distal portion of the catheter, wherein the distal end opening is coaxial with a cylinder opening defined by a cylinder wall of a cylinder, wherein the cylinder is connected to a distal terminal end of the helix member;
fragmenting occlusive material with a plurality of cantilevered blade members that extend inwardly from the cylinder wall as the occlusive material enters the cylinder opening; and
transporting the occlusive material from the cylinder opening to a proximal portion of the catheter assembly via the helix member.

21. The method of claim 20, wherein the distal portion defines a lateral opening, positioned distal to the cylinder.
